SEQUENCE LISTING (8 sequences)

SEQ ID NO: 1 - DNA, 20 bp, Artificial Sequence
  Description of Artificial Sequence Synthetic primer
cgccacctattttgccgatg

SEQ ID NO: 2 - DNA, 20 bp, Artificial Sequence
  Description of Artificial Sequence Synthetic primer
gtactcagacacagaccggc

SEQ ID NO: 3 - DNA, 20 bp, Artificial Sequence
  Description of Artificial Sequence Synthetic primer
cagcaccatcaccaacatgc

SEQ ID NO: 4 - DNA, 20 bp, Artificial Sequence
  Description of Artificial Sequence Synthetic primer
ctgcacatgtcctggatcga

SEQ ID NO: 5 - DNA, 20 bp, Artificial Sequence
  Description of Artificial Sequence Synthetic primer
ctcaaggtgctggcatggta

SEQ ID NO: 6 - DNA, 20 bp, Artificial Sequence
  Description of Artificial Sequence Synthetic primer
cagctgccactgttggtttc

SEQ ID NO: 7 - DNA, 20 bp, Artificial Sequence
  Description of Artificial Sequence Synthetic primer
agagcaagaaccagacctac

SEQ ID NO: 8 - DNA, 20 bp, Artificial Sequence
  Description of Artificial Sequence Synthetic primer
gacggaacatagcagtgaac

(19) United States
(12) Patent Application Publication
(10) Pub. No.: US 2022/0249576 A1
(43) Pub. Date: Aug. 11, 2022

(54) METHOD OF DISRUPTING MYCOTOXIN SYNTHESIS USING A COMPOSITION COMPRISING VIBRIO GAZOGENES

(71) Applicant: Mycologics LLC, Alexandria, VA (US)

(72) Inventor: Anindya CHANDA, Wilmington, NC (US)

(21) Appl. No.: 17/592,575

(22) Filed: Feb. 4, 2022

Related U.S. Application Data
(60) Provisional application No. 63/147,447, filed on Feb. 9, 2021.

(51) Int. Cl. A61K 35/74; A01N 63/20; A61P 31/10

(52) U.S. Cl. CPC A61K 35/74; A61P 31/10; A01N 63/20

(57) ABSTRACT

A method of inhibiting mycotoxin synthesis in filamentous fungi comprises contacting the filamentous fungi with a composition comprising Vibrio gazogenes, ATCC. Additionally, infections caused by filamentous fungi can be inhibited by contacting the filamentous fungi with a composition comprising Vibrio gazogenes, ATCC 43942. Endosomal function in filamentous fungi can be disrupted by contacting the filamentous fungi with a composition comprising Vibrio gazogenes, ATCC 43942. Hyphal fusion formation can be restricted in filamentous fungi by contacting the filamentous fungi with a composition comprising Vibrio gazogenes, ATCC 43942.

### CROSS-REFERENCE TO RELATED APPLICATION

This application claims the benefit of U.S. Provisional Patent Application No. 63/147,447, filed on Feb. 9, 2021, the entire content of which is incorporated herein by reference.

### SEQUENCE LISTING

The instant application contains a Sequence Listing which has been submitted electronically in ASCII format and is hereby incorporated by reference in its entirety. Said ASCII copy, created on Mar. 29, 2022, is named 1247_2_UTIL_SL.txt and is 2,117 bytes in size.

### BACKGROUND OF THE INVENTION

Mycotoxins (fungal toxins) are toxic compounds that are naturally produced by certain types of fungi. Fungi that can produce mycotoxins grow on various types of substrates that includes a variety of food and feed crops. Additionally, mycotoxin producers can also grow in building materials such as dirty air conditioning vents and filters and have been associated with deteriorated indoor air quality during the aftermath of water damage to interiors. Most mycotoxins are chemically stable and survive food processing. Hundreds of different mycotoxins have been identified, but the most commonly observed mycotoxins that present a concern to human health and livestock include aflatoxins, ochratoxin A, patulin, fumonisins, zearalenone and nivalenol/deoxynivalenol.

Mycotoxins appear in the food chain as a result of fungal infection of crops both before and after harvest. The effects of some food-borne mycotoxins are acute with symptoms of severe illness appearing quickly after consumption of food products contaminated with mycotoxins. Other mycotoxins occurring in food have been linked to long-term effects on health, including the induction of cancers and immune deficiency. Of the several hundred mycotoxins identified so far, about a dozen have gained the most attention due to their severe effects on human health and their occurrences in food. Aflatoxins are among the most concerning mycotoxins and are produced by certain fungi (Aspergillus flavus and Aspergillus parasiticus) which grow in soil, decaying vegetation, hay, and grains. Crops that are frequently affected by Aspergillus spp. include cereals (corn, sorghum, wheat and rice), oilseeds (soybean, peanut, sunflower and cotton seeds), spices (chili peppers, black pepper, coriander, turmeric and ginger) and tree nuts (pistachio, almond, walnut, coconut and Brazil nut). The toxins can also be found in the milk of animals that are fed contaminated feed.

In order to infect hosts and make mycotoxins, fungi, in particular, filamentous fungi use endosomes to organize a variety of functions, including transport of lipids, proteins, messenger RNAs, ribosomes and metabolites. Endosomes aid with hyphal fusion. Broadly speaking, hyphal fusion generally involves the formation of channels between fungal hyphae, eventually resulting in a fungal colony being a complex interconnected network of hyphae. Hyphal fusion is a fundamental process used in tissue development, heterokaryon formation, nutrition communication and eventually the development of the entire mycelium colony.

Endosomes and endocytosis (an endosome mediated process for cellular uptake) facilitate growth and hyphal elongation, which are integral processes for hyphal fusion and fungal colony expansion. Endosomes also organize biosynthesis of mycotoxins by transporting the necessary proteins and metabolites to the location of toxin synthesis in cells and eventually hosting the process of toxin biosysnthesis.

Accordingly, technologies that can disrupt endosomal function can inhibit mycotoxin production and thus, reduce or prevent fungal infection in crops, animals and humans.

### SUMMARY OF THE INVENTION

In a first aspect of the invention, a method of inhibiting mycotoxin synthesis in filamentous fungi comprises contacting the filamentous fungi with a composition comprising Vibrio gazogenes, ATCC 43942 thereby inhibiting mycotoxin synthesis in the filamentous fungi.

In another aspect of the invention, a method of disrupting endosomal function in filamentous fungi comprises contacting the filamentous fungi with a composition comprising Vibrio gazogenes, ATCC 43942 thereby disrupting endosomal function in the fungal cell.

In yet another aspect of the invention, a method of restricting hyphal fusion formation in filamentous fungi comprises contacting the filamentous fungi with a composition comprising Vibrio gazogenes, ATCC 43942 thereby restricting hyphal fusion formation in the fungal cell.

In a feature of the aspects, the methods may further comprise heating the composition comprising Vibrio gazogenes, ATCC 43942 at a suitable temperature and for a suitable time prior to contacting the composition with filamentous fungi to kill the Vibrio gazogenes, ATCC 43942.

It is to be understood that both the foregoing general description of the invention and the following detailed description are exemplary but are not restrictive of the invention.

### BRIEF DESCRIPTION OF THE FIGURES

A more complete appreciation of the invention and many of the attendant advantages thereof will be readily obtained as the same becomes better understood by reference to the following detailed description when considered in connection with the accompanying drawings. The patent or application file contains at least one drawing executed in color. Copies of this patent or patent application publication with color drawings) will be provided by the Office upon request and payment of the necessary fee. In the Figures, Vibrio gazogenes may be abbreviated as Vg.

FIG. 1A illustrates the influence of V. gazogenes uptake on hyphal fusion. FIG. 1A is a flowchart of the method (experimental framework) to measure hyphal fusion.

FIG. 1B is a chart showing the number of heterokaryotic colonies formed after the ΔpyrG and ΔargB strains were allowed to cross. The statistical significance of two-tailed p-values between the outcomes of control vs. treated was determined using an unpaired t-test with sample size n=3 and p<0.05 set as level of significance. ** statistically significant with p<0.001.

FIG. 2 illustrates the influence of treatment with an exemplary Vibrio gazogenes composition on the expression of aflatoxin genes. FIG. 2 is a chart showing a comparison of expressions of aflatoxin genes af1C, af1D, af1M and af1R and the house-keeping gene β-tubulin in the untreated control and Vibrio gazogenes composition-treated mycelia at 40 h post inoculation. Error bars, SEM, from triplicate experiments. Statistical significance of two-tailed p-values determined using an unpaired t-test for n=3. (**, p<0.05).

FIG. 3A is a series of photographs of flasks with fungal growth cultures at 24 h and 40 h post inoculation (i.e. start of culture).

FIG. 3B is a series of photographs of harvested fungal cultures harvested at 40 h post inoculation.

FIG. 4A is a flow chart illustrating the theory that the fungal mycelia internalizes the Vibrio gazogenes components that are naturally red in color red.

FIG. 4B is a series of photographs showing the influence of filipin and natamycin on uptake of naturally red colored components of Vibrio gazogenes and aflatoxin production.

FIG. 5 is a schematic diagram illustrating a theory of how the composition comprising Vibrio gazogenes inhibits aflatoxin biosynthesis and hyphal fusion in the Aspergillus flavus model.

### DETAILED DESCRIPTION

Described herein is a composition comprising Vibrio gazogenes, ATCC 43942 and a method of using the composition to disrupt endosomal function in filamentous fungi. Through disruption of endosomal function, hyphal fusion is restricted and mycotoxin synthesis is reduced or inhibited. In embodiments, the composition comprising Vibrio gazogenes, ATCC 43942 is heated prior to contact with filamentous fungi in order to kill the bacterium. The composition comprising Vibrio gazogenes, ATCC 43942 is effective in disrupting endosomal function in filamentous fungi even when the bacterium is dead. As used herein, the term “Vibrio gazogenes” means Vibrio gazogenes, ATCC 43942. Additionally, Vibrio gazogenes may be abbreviated as Vg or Vg herein.

Effectiveness of disrupting endosomal function has been demonstrated on Aspergillus flavus, which is a fungus that produces aflatoxin. Aflatoxin production by Aspergillus flavus has been widely used as a biological model to study the mechanisms of mycotoxin production by fungi. Additionally Aspergillus flavus has been widely used as a fungal growth model, while identifying natural compounds with antifungal activity. For example, A. flavus has been used as a biological model to study mycotoxin production and as a fungal model to identify antifungal natural compounds.

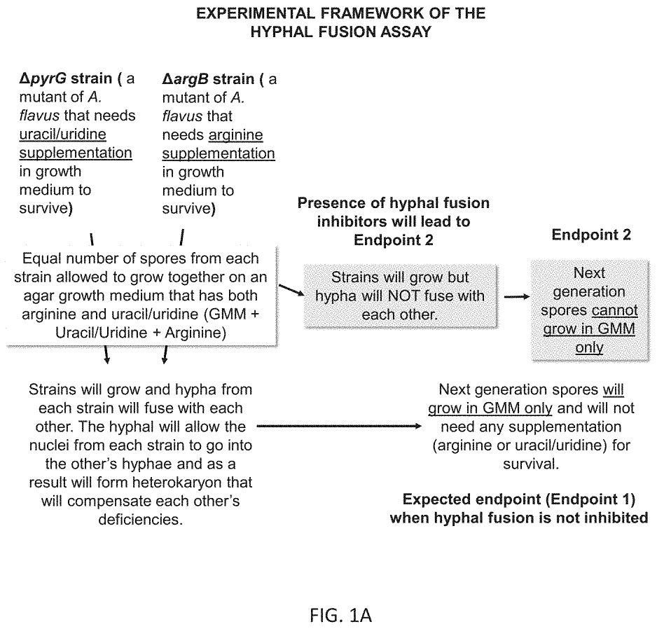

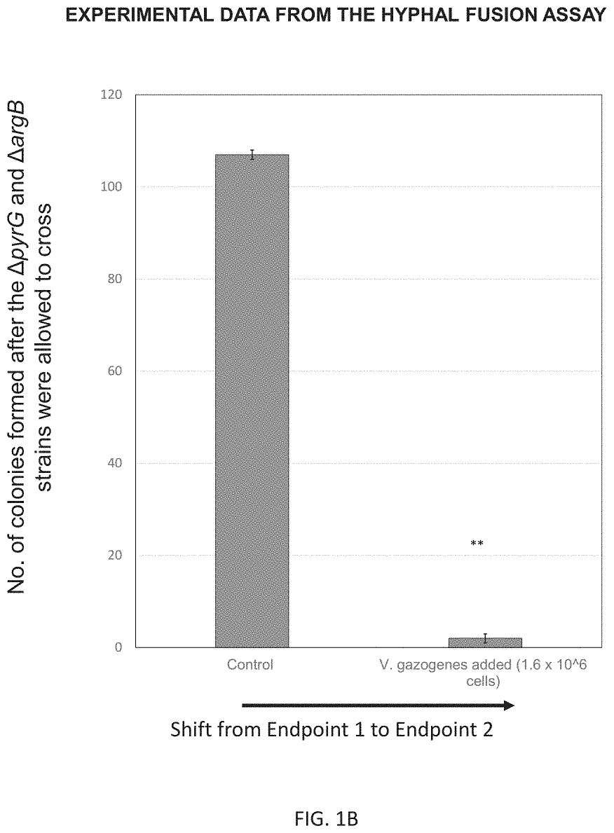

As will be described in greater detail below, testing using a hyphal fusion assay was performed to evaluate whether a composition comprising dead Vibrio gazogenes was able to impede fungal colony development. The assay is based on the rationale that fungal heterokaryon formation requires hyphal fusion formation. Thus, any impediment to hyphal fusion formation reduces, inhibits or prevents heterokaryon formation. FIG. 1a is a schematic flow diagram of the hyphal fusion assay.

The composition comprising Vibrio gazogenes can inhibit mycotoxin synthesis. In testing, the composition inhibited synthesis of at least three mycotoxins: Aflatoxin B (AFB1), Aflatoxin B2 (AFB2) and Cyclopiazonic acid (CPA). The results and mechanism of action suggest that the composition comprising Vibrio gazogenes is a broad spectrum mycotoxin inhibitor rather than a specific mycotoxin inhibitor.

To enable inhibition of hyphal fusion, the composition comprising Vibrio gazogenes is able to disrupt endosomal functioning within the hyphae. When filamentous fungi are contacted with the composition comprising dead Vibrio gazogenes, the composition internalizes within hyphae of the fungi. At least part of the internalizing occurs via endocytosis, which is a process that leads to the formation of early endosomes.

The presence of at least a portion of the composition comprising Vibrio gazogenes in endosomes of the fungi disrupts synthesis of mycotoxins in the fungi and the process of hyphal fusion, which is extremely important in reducing or inhibiting the start and spread of dangerous infections caused by filamentous fungi.

### EXAMPLES

Evaluation of a composition comprising dead Vibrio gazogenes was performed using the filamentous fungus Aspergillus flavus NRRL 3357, which is a well-established biological model for studying (a) how fungal pathogens grow on substrates and infect host cells and (b) how fungi biosynthesize mycotoxins, such as aflatoxin.

### Example 1

An exemplary composition comprising dead Vibrio gazogenes was prepared as follows. Vibrio gazogenes, ATCC 43942, was grown in Difco Marine Broth 2216 (BD Biosciences, Sparks, Md., USA) at 25° C. in a shaking incubator (250 rpm) in the dark for 24 h prior to harvesting the cells for the interaction experiments. Harvested cells were pelleted by centrifugation at 4000 g for 15 minutes at room temperature and then resuspended in a solution comprising of 2% (w/v) commercially obtained yeast extract and 6% (w/v) sucrose and adjusted to pH 5.8. The final V. gazogenes cell concentration in the solution was 1.6×10⁷ cells/mL. The solution with the dead Vibrio gazogenes was heated at 100° C. for 15 minutes to kill the V. gazogenes cells in the solution. Suitable heating temperatures and times include any temperature and time that is effective in killing the bacterium.

### Example 2

Testing was performed to evaluate the composition comprising dead Vibrio gazogenes for impeding fungal colony development. A known hyphal fusion assay was used. The assay is based on the rationale that fungal heterokaryon formation requires hyphal fusion formation. Thus, any impediment to hyphal fusion formation will inhibit or prevent heterokaryon formation.

In the assay, equal numbers of conidia from pyrG auxotroph (TJES 19.1) and argB auxotroph (TJES 20.1) were mixed together and grown on GMM agar supplemented with uracil/uridine and arginine. After 5 days, mixed conidia were spread onto GMM agar plates lacking supplementation where only conidia generated from heterokaryons could grow. The plates of mixed cultures were incubated at 29° C. for 3 days. The control plate contained only mixed conidia. The test plate contained mixed conidia and a composition comprising dead Vibrio gazogenes. As shown in FIG. 1A, the expected endpoint for control plate is Endpoint 1 in which after successful hyphal fusion, the next generation spores will not need any uracil/uridine and arginine supplementation and will be able to grow on unsupplemented GMM plates. On the contrary, any impediment to hyphal fusion in a test plate would shift the endpoint to Endpoint 2, which due to disruption of hyphal fusion, the next generation spores will lack the heterokaryon and will be unable to grow on unsupplemented GMM plates. FIG. 1B shows the representative photograph from a control plate and a representative photograph from the test plate along with a chart showing the number of heterokaryotic colonies formed from the control and the test plates. In the chart, **P<0.001.

As shown in FIG. 1B, crosses between TJES 19.1 and TJES 20.1 in the presence of a composition comprising dead Vibrio gazogenes, almost completely inhibited (by >95%) heterokaryon formation. Experimentation results showed that hyphal elongation terminates earlier upon V. gazogenes treatment leading to early spore development. Additionally, treated colonies also showed lesser branching. Radial growth of V. gazogenes treated and untreated control colonies were compared by measuring the average distance of the tip from the center of the colony following growth on PDA for 5 days at 30° C. in the dark. A total of 10 measurements were taken from each colony and the average±SD was determined for each sample. Quantitative comparison of branching was conducted using a visualization scoring method to quantify the differences that were visible under the microscope as follows. Twenty investigators were asked to assess a control (untreated) A. flavus colony and a V. gazogenes-treated colony. In a blinded fashion, they were asked to assign a score of 0-10, with 0 representing no branching and 10 representing the extent of branching observed in the control. Statistical significance of two-tailed p-values between the outcomes of control vs. treated was determined using an unpaired t-test with sample size n=3 and p<0.05 set as level of significance

### Example 3

Experiments were performed by growing A. flavus fungus in a liquid growth medium. 1 mL of the exemplary composition comprising Vibrio gazogenes prepared in Example 1 was added to 100 mL liquid culture at the start of fungal growth. Aflatoxin analysis by UPLC showed that samples treated with the exemplary composition did not produce any AFB1, AFB₂ or CPA, while the control strain under the growth conditions of the experiment produced ˜32±0.7 ng AFB1, ˜4.7±0.5 ng AFB2 and 65±0.04 μg CPA per gm of fungal mycelia.

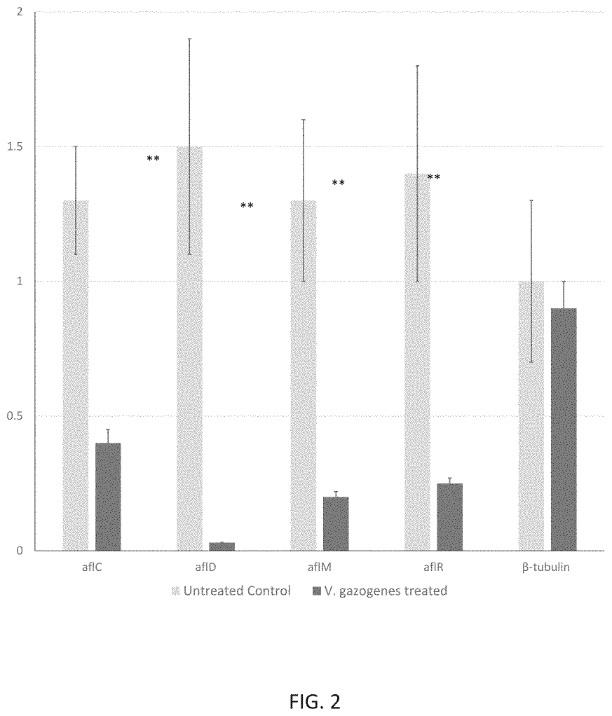

To evaluate if the observed block of mycotoxin production upon treatment with the exemplary composition occurred at the level of gene regulation, gene expression levels of the aflatoxin pathway regulator (aflR) and three aflatoxin pathway genes, pksA (aflC), nor-1 (aflD), and ver-1 (aflM) were compared for control and the exemplary composition. FIG. 2 is a chart showing the influence of treatment with the exemplary composition on the expression of aflatoxin genes. In particular, FIG. 2 shows the comparison of expressions of aflatoxin genes aflC, aflD, aflM and aflR and the house-keeping gene β-tubulin in the untreated control and exemplary composition treated mycelia at 40 h post inoculation. In FIG. 2, the error bars are from SEM from triplicate experiments. Statistical significance of two-tailed p-values were determined using an unpaired t-test for n=3. (**, p<0.05). As can be seen, treatment with the exemplary composition resulted in statistically significant decreases in the expression of all four aflatoxin genes, while no significant expression change was observed for the β-tubulin gene, which is used as a housekeeping gene in this study.

### Example 4

Testing was performed to evaluate whether the exemplary composition of Example 1 was able to disrupt endosomal functioning within the hyphae of filamentous fungi. The testing results indicate that the exemplary composition internalized within hyphae and the internalization, at least in part, occurred via endocytosis, a process that leads to the formation of early endosomes.

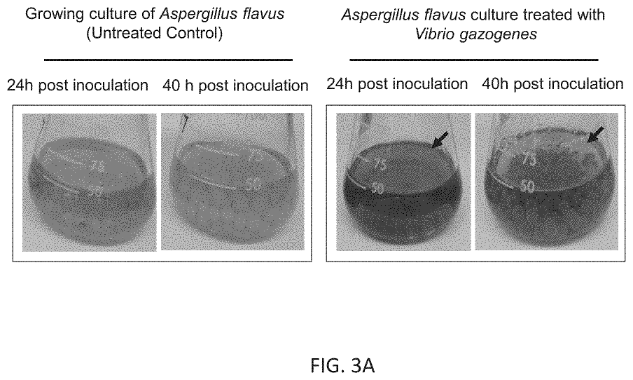

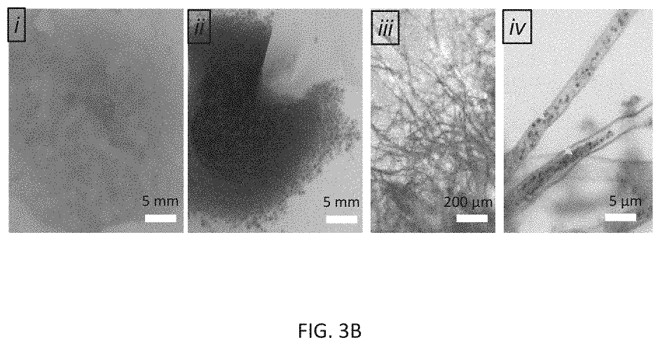

The exemplary composition was red in color. When the exemplary composition was applied to the A. flavus culture, the fungal mycelia consistently turned red (i.e., the same color as the exemplary composition), and the medium lost the red color, which indicated that the exemplary composition was taken up by the growing fungal culture. Additionally, Bright-field microscopy indicated that the exemplary composition internalized in hyphae at discrete locations. FIGS. 3A and 3B provide evidence Vibrio gazogenes uptake by A. flavus hypha during Vibrio gazogenes treatment. FIG. 3A is a series of photographs of flasks with fungal growth cultures at 24 h and 40 h post inoculation (i.e. start of culture). The left panel includes photographs of flasks with untreated (control) culture. The right panel includes photographs of flasks with Vibrio gazogenes treated cultures. The arrows point to the colors of growth medium at 24 h and 40 h post inoculation. When the uptake of Vibrio gazogenes is still ongoing (for example at 24 h time point), the growth medium is still red in color (representing the natural color of Vibrio gazogenes). When the uptake of Vibrio gazogenes by the fungal culture is complete (for example at 40 h time point), the growth medium loses its red color, and the red color gets transferred to the fungal pellets. FIG. 3B is a series of photographs of harvested fungal cultures harvested at 40 h post inoculation. Panel (i) shows untreated (control) fungal culture. Panels (ii)-(iv) show vibrio gazogenes treated fungal cultures, where (iii) shows a magnified image a mycelial section showing evidence of reddish components from Vibrio gazogenes in hyphal sections (indicated with arrows), and (iv) shows a magnified image of hyphae showing evidence of reddish colored components (of Vibrio gazogenes) in fungal hyphae.

### Example 5

Testing was performed to evaluate whether the exemplary composition was at least partially internalized by endocytosis. The testing evaluated whether the established endocytosis inhibitors filipin and natamycin inhibited uptake of the exemplary composition, and in doing so, reversed the aflatoxin inhibition caused by the exemplary composition thereby leading to an increase in aflatoxin production.

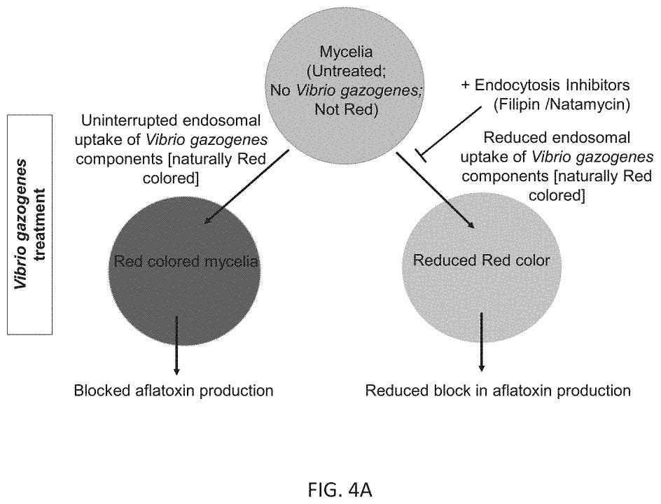

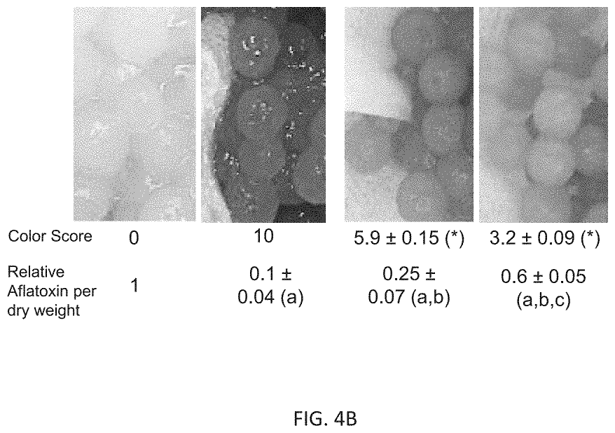

Using a known protocol for filipin and natamycin application, the effect of filipin and natamycin on the exemplary composition uptake and aflatoxin production was examined. FIGS. 4A and 4B show the effect of endocytosis inhibitors on uptake of naturally red colored components of Vibrio gazogenes and aflatoxin production. FIG. 4A is a flow chart illustrating the theory that the fungal mycelia internalizes the Vibrio gazogenes components that are naturally red in color red. The mechanism of uptake is, at least in part, the endosomal uptake process (also termed ‘endocytosis’). This concept was tested by using two different endocytosis inhibitors (Filipin and Natamycin). The hypotheses tested were: (1) Application of endocytosis inhibitors reduces uptake of the naturally red colored Vibrio gazogenes components and (2) Reduction of the uptake of the naturally red colored Vibrio gazogenes components also increase aflatoxin production by the fungus. FIG. 4B is a series of photographs showing the influence of filipin and natamycin on uptake of naturally red colored components of Vibrio gazogenes and aflatoxin production. A comparative color scoring technique was used to compare the “redness” of the mycelial pellets that were representative of uptake of naturally red colored components of Vibrio gazogenes by 40 h mycelia. Twenty investigators in blinded fashion assigned scores within a scale of 0 to 10, where 0 represented mycelial color of wild-type (WT) and 10 represented color mycelial color of WT+Vg. Statistical significance of the difference in color score was determined using an unpaired t-test (n=20); (*) P<0.05 and considered statistically significantly different. Total aflatoxin production (ng/g) per gram of mycelia at 40 h time point is shown. Statistical difference was determined using an unpaired t-test (n=3) and P<0.05 was considered statistically different. The letter a indicates statistical difference in comparison with WT. The letter b indicates statistical difference in comparison with WT+Vibrio gazogenes. The letter c indicates statistical difference in comparison with WT+Vibrio gazogenes (+Filipin). Concentrations of filipin (3 μM) and natamycin (6 μM) were chosen based on a previous study (van Leeuwen et al., 2009).

Aflatoxin levels correlated well with the inhibition levels the exemplary composition uptake. While both inhibitor treatments were able to increase aflatoxin production compared to the untreated control (A. flavus+the exemplary composition), natamycin resulted in a significantly greater increase of aflatoxin levels compared to the filipin treated samples.

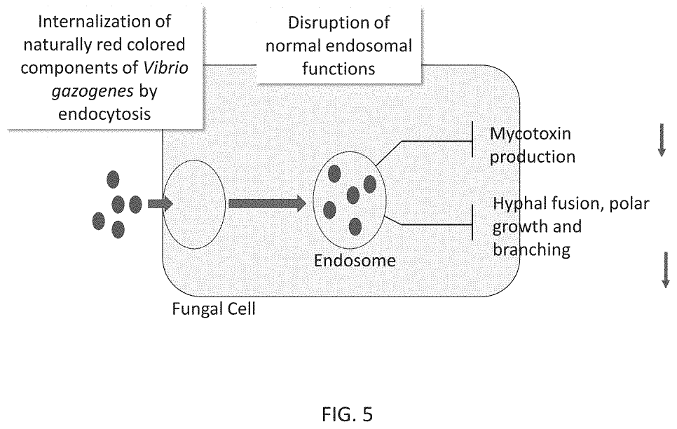

FIG. 5 is a schematic diagram illustrating a theory of how the composition comprising Vibrio gazogenes inhibits aflatoxin biosynthesis and hyphal fusion in the Aspergillus flavus model. According to this model, the naturally red colored components of Vibrio gazogenes internalize via endocytosis and localize in endosomes where they interfere in the normal endosomal functions. The disruption of normal endosomal functions, as a result, restricts aflatoxin biosynthesis and inhibits hyphal fusion resulting in restricted colony development (reduced hyphal elongation and branching).

Testing with endocytosis inhibitors confirmed that at least a part of the composition components reach endosomes.

In the Examples, the following specific materials and methods were used.

### List of Fungal Strains

| Strain | Genotype | Strain source |
|---|---|---|
| NRRL 3357 | A. flavus Wild type | [18] |
| TJES 19.1 | Δku70, pyrG⁻ | [9] |
| TJES 20.1 | Δku70, ΔargB:: A. fumigatus pyrG, pyrG⁻ | [9] |
| TXZ9.16 | Δku70, ΔhamI:: A. flavus argB, pyrG⁻ | [9] |

Fungal dry weight measurements. Fungal dry weight was measured by collecting mycelia from the growth medium using Miracloth (Millipore, Billerica, Mass.) and then drying the mycelial mass in an oven at 80° C. for 6 h. Weight difference before and after drying was recorded as the dry weight.
RNA extraction, purification and transcript analysis by qRT-PCR. Total RNA was extracted from fungal cells harvested at 24 h, 30 h, and 40 h post-inoculation using a TRIzol-based (Sigma, Carlsbad, Calif., USA) method as described previously [20]. Within 24 h of extraction, RNA purification was performed using a Qiagen RNEasy Cleanup Kit (Qiagen, Valencia, Calif., USA), as per the kit's instructions, and samples were stored at −80° C. Total RNA was then used for cDNA synthesis using iScript™ cDNA Synthesis Kit (BioRad Laboratories, Hercules, Calif., USA) as per manufacturer's instructions. All samples were checked for concentration and purity after each step using a NanoDrop 2000 Spectrophotometer (Thermo-Fisher Scientific, Waltham, Mass., USA). All cDNA samples were stored at −20° C. until subsequent RTPCR quantification. Quantitative RT-PCR (qRT-PCR) was performed using SYBR green I chemistry and CFX96 Real-Time PCR detection system (Bio-Rad). A pre-incubation at 95° C. for 3 min, dye activation at 95° C. for 10 s, primer annealing at 55° C. for 30 s, elongation at 55° C. for 50 s followed by a dissociation curve from 65° C. to 95° C. for 30 min (with 0.5° C. increments). The primers used for qRT-PCR are provided in Table 2. Gene expression was normalized by ΔΔC_{T} analysis [21] to A. flavus β-tubulin gene (AFLA_068620) using the gene expression analysis software package (Bio-Rad CFX Maestro) of the Bio-Rad CFX96.

Oligonucleotide primers used for the qRT-PCR

| Genes | Primers |
|---|---|
| pksA (aflC) | F 5′- CGCCACCTATTTTGCCGATG-3′ |
| | (SEQ ID NO: 1) |
| | R 5′-GTACTCAGACACAGACCGGC-3′ |
| | SEQ ID NO: 2) |
| nor-1 (aflD) | F 5′- CAGCACCATCACCAACATGC -3′ |
| | (SEQ ID NO: 3) |
| | R 5′- CTGCACATGTCCTGGATCGA-3′ |
| | (SEQ ID NO: 4) |
| ver-1 (aflM) | F 5′- CGCCACCTATTTTGCCGATG-3′ |
| | (SEQ ID NO: 1) |
| | R 5′- GTACTCAGACACAGACCGGC-3′ |
| | (SEQ ID NO: 2) |
| aflR | F 5′- CTCAAGGTGCTGGCATGGTA-3′ |
| | (SEQ ID NO: 5) |
| | R 5′- CAGCTGCCACTGTTGGTTTC-3′ |
| | (SEQ ID NO: 6) |
| B-tubulin | F5′-AGAGCAAGAACCAGACCTAC-3′ |
| | (SEQ ID NO: 7) |
| | R5′-GACGGAACATAGCAGTGAAC-3′ |
| | (SEQ ID NO: 8) |

Fungal Imaging experiments. For light imaging experiments, A. flavus spores (˜10² spores) were inoculated in 20 μL of fungal growth medium on a glass-bottom dish with Vibrio gazogenes composition added to the growth medium. For bright-field microscopy a widefield microscope (Finite Optical System, Fischer Scientific) was used. A. flavus colonies imaged for hyphal branching and radial growth were grown on solid agar medium with or without Vibrio gazogenes composition treatment and were visualized using a stereomicroscope (EZ4 W, Leica microsystems).

Hyphal fusion assay. An established assay described previously [9] was used. Briefly, equal concentrations of the A. flavus pyrG auxotrophic strain (TJES 19.1) and argB auxotrophic strain (TJES 20.1) were mixed and spotted onto a GMM agar media containing arginine (1 g/L) and uracil (5 mM)/uridine (5 mM) with or without Vibrio gazogenes composition. After incubation at 29° C. for 5 days, the newly formed conidia were collected in Phosphate Buffered Saline, and 10⁵ conidia were spread onto the surface of GMM+0.25% Triton X-100 agar plates (the 0.25% Triton X-100 restricts colony diameter to help with precise colony counts). Heterokaryotic colonies were counted after incubation for 3 days at 29° C. A cross of the hyphal anastomosis mutant, ΔhamI-TXZ9.16 with TJES 20.1 was used as a negative control.

Radial growth and hyphal branching assays. Determination of radial growth of Vg composition-treated and control colonies was conducted by measuring using a ruler, the average distance of the tip from the center of the colony following growth on potato dextrose agar (PDA) medium for 5 days at 30° C. in the dark. A total of 10 measurements were taken from each colony and the average±SD was determined for each sample. For a visual comparison of the extent of branching between the Vibrio gazogenes composition-treated and untreated colonies, the colonies grown on PDA were observed under a stereomicroscope (EZ 4w, Leica Microsystems). A visualization scoring method was developed to quantify the differences that were visible under the microscope as follows. Twenty investigators were asked to assess a control (untreated) A. flavus colony and a Vibrio gazogenes composition-treated colony. In a blinded fashion, they were asked to assign a score of 0-10, with 0 representing no branching and 10 representing the extent of branching observed in the control.

Treatment with endocytosis inhibitors. Two polyene endocytosis inhibitors, filipin and natamycin (both purchased from Sigma) were used to study their effect on Vibrio gazogenes composition uptake and aflatoxin production. Stock solution (1 mmol L⁻¹) of the inhibitors were made with DMSO. For treatment, we used filipin at 3 μmol L⁻¹ and natamycin at 6 μmol L⁻¹. These doses correspond to the minimal inhibitory concentrations (MICs) previously observed for the disruption of Aspergillus endosomal development [17].
Color scoring assay for evaluation of composition comprising Vg composition uptake. Since mycelial uptake of red-colored Vibrio gazogenes composition correlates with the transition of normally non-pigmented fungal hyphae to a reddish color upon complete uptake at 40 h, the effect of the inhibitors on Vibrio gazogenes composition uptake was assessed by visual monitoring of the mycelia and by comparing its color to the control mycelia. A less intense red color corresponded to less uptake. To determine whether the inhibition of Vibrio gazogenes composition uptake was statistically significant, a blinded visual scoring method was developed to conduct a quantitative comparison of Vibrio gazogenes composition uptake in the presence and absence of endocytosis inhibitors. A total of 20 separate examiners, who had no prior knowledge of the samples, were asked to provide a color intensity score to 40 h mycelial pellets representing untreated control (displaying normal Vibrio gazogenes composition uptake) and the natamycin and filipin treated pellets (displaying Vibrio gazogenes composition uptake in the presence of these inhibitors). Examiners were asked to provide a color score within the scale of 0 to 10, where 0 represents no red color in mycelial pellets, as seen in 40 h untreated pellets (negative control), and 10=totally red mycelial pellet, as seen in Vibrio gazogenes composition-treated A. flavus (positive control). The choice of 40 h old pellets for color scoring was based on the rationale that mycelia show the highest color intensity (corresponding to an almost complete Vibrio gazogenes composition uptake) at this time point under the culture conditions used for the experiments. A higher red color score, therefore, represented higher Vibrio gazogenes composition uptake.
Mycotoxin measurements. Determination of aflatoxin and cyclopiazonic acid production was conducted using fungal broths concentrated in vacuo. For aflatoxin determination, an extraction with ethyl acetate/acetone (1:1)/0.1% formic acid (20 mL) for 24 h at room temperature was performed as described previously [22]. The extract was filtered, and the filtrate was concentrated under nitrogen to dryness, redissolved in acetonitrile (˜1 mg/mL), filtered through a Spin-X centrifuge spin tube filter (Costar), and then analyzed on a Waters Acquity UPLC system (40% MeOH in water, BEH C18 1.7 μm, 2.1×50 mm column) using fluorescence detection (ex=365 nm, em=440 nm). For cyclopiazonic acid determination a methanol extraction was conducted with ethanol (5 mg/mL) and filtered for analysis on the Waters Acquity UPLC system using PDA detection and the following gradient solvent system (solvent A: 0.1% formic acid in water; solvent B: 0.1% formic acid in acetonitrile): 5% B (0-2.5 min), gradient to 25% B (2.5-3 min), gradient to 100% B (3-10 min), 100% B (10-15 min), then column equilibration 5% B (15.1-20.1 min). Cyclopiazonic acid standard was used to confirm identity (CPA, rt=4.10 min), while Aflatoxin B1 and B2 was identified based on UV spectra (λₘₐₓ 231.1, 282.6, 292.0 (sh) nm.). Aflatoxin B₁, B₂ and CPA standards were purchased from Sigma. Aldrich (St. Louis, Mo., USA). Three biological replicates of each culture were collected for analysis of secondary metabolites.
Statistical analysis. All statistical tests were performed using GraphPad Prism Software (GraphPad, La Jolla, Calif., USA). Statistical analyses to determine for statistical significance of differences between control versus experimental groups were determined using one-way ANOVA. An unpaired t-test was used to determine the gene expression effects of Vg composition on A. flavus compared to the untreated samples. Significance was set at p<0.05.

### REFERENCES

1. Fischer, M. S. and N. L. Glass, Communicate and Fuse: How Filamentous Fungi Establish and Maintain an Interconnected Mycelial Network. Frontiers in Microbiology, 2019. 10(619).
2. Riquelme, M., et al., Fungal Morphogenesis, from the Polarized Growth of Hyphae to Complex Reproduction and Infection Structures. Microbiol Mol Biol Rev, 2018. 82(2).
3. Fu, C., et al., Identification and characterization of genes required for cell-to-cell fusion in Neurospora crassa. Eukaryot Cell, 2011. 10(8): p. 1100-9.
4. Glass, N. L., et al., Hyphal homing, fusion and mycelial interconnectedness. Trends Microbiol, 2004. 12(3): p. 135-41.
5. Riquelme, M., et al., Spitzenkorper localization and intracellular traffic of green fluorescent protein-labeled CHS-3 and CHS-6 chitin synthases in living hyphae of Neurospora crassa. Eukaryot Cell, 2007. 6(10): p. 1853-64.
6. Sanchez-Leon, E., et al., The Rab GTPase YPT-1 associates with Golgi cisternae and Spitzenkorper microvesicles in Neurospora crassa. Mol Microbiol, 2015. 95(3): p. 472-90.
7. Upadhyay, S. and B. D. Shaw, The role of actin, fimbrin and endocytosis in growth of hyphae in Aspergillus nidulans. Mol Microbiol, 2008. 68(3): p. 690-705.
8. Zhao, X., et al., A Cellular Fusion Cascade Regulated by LaeA Is Required for Sclerotial Development in Aspergillus flavus. Front Microbiol, 2017. 8: p. 1925.
9. Daskalov, A., et al., Molecular Mechanisms Regulating Cell Fusion and Heterokaryon Formation in Filamentous Fungi. Microbiol Spectr, 2017. 5(2).
10. Chanda, A., et al., A key role for vesicles in fungal secondary metabolism. Proc Natl Acad Sci USA, 2009. 106(46): p. 19533-8.
11. Lim, F. Y. and N. P. Keller, Spatial and temporal control of fungal natural product synthesis. Nat Prod Rep, 2014. 31(10): p. 1277-86.
12. Linz, J. E., et al., Proteomic and biochemical evidence support a role for transport vesicles and endosomes in stress response and secondary metabolism in Aspergillus parasiticus. J Proteome Res, 2012. 11(2): p. 767-75.
13. Amaike, S. and N. P. Keller, Aspergillus flavus. Annual Review of Phytopathology, 2011. 49(1): p. 107-133.
14. Scheidegger, K. A. and G. A. Payne, Unlocking the Secrets Behind Secondary Metabolism: A Review of Aspergillus flavus from Pathogenicity to Functional Genomics. Journal of Toxicology: Toxin Reviews, 2003. 22(2-3): p. 423-459.
15. Zhao, X., et al., A Cellular Fusion Cascade Regulated by LaeA Is Required for Sclerotial Development in Aspergillus flavus. Frontiers in Microbiology, 2017. 8(1925).
16. Van Leeuwen, M., E. Golovina, and J. Dijksterhuis, The polyene antimycotics nystatin and filipin disrupt the plasma membrane, whereas natamycin inhibits endocytosis in germinating conidia of Penicillium discolor. Journal of applied microbiology, 2009. 106(6): p. 1908-1918.
17. Payne, G. A., et al., Cloning of the afl-2 gene involved in aflatoxin biosynthesis from Aspergillus flavus. Appl Environ Microbiol, 1993. 59(1): p. 156-62.
18. Maggio-Hall, L. A., R. A. Wilson, and N. P. Keller, Fundamental contribution of beta-oxidation to polyketide mycotoxin production in planta. Mol Plant Microbe Interact, 2005. 18(8): p. 783-93.
19. Gummadidala, P. M., et al., Aflatoxin-Exposure of Vibrio gazogenes as a Novel System for the Generation of Aflatoxin Synthesis Inhibitors. Front Microbiol, 2016. 7: p. 814.
20. Livak, K. J. and T. D. Schmittgen, Analysis of relative gene expression data using real-time quantitative PCR and the 2-ΔΔCT method. methods, 2001. 25(4): p. 402-408.
21. Cary, J. W., et al., The Aspergillus flavus Homeobox Gene, hbx1, is Required for Development and Aflatoxin Production. Toxins (Basel), 2017. 9(10).

That which is claimed:

1. A method of inhibiting mycotoxin synthesis in filamentous fungi, comprising contacting the filamentous fungi with a composition comprising Vibrio gazogenes, ATCC 43942 thereby inhibiting mycotoxin synthesis in the filamentous fungi.

2. A method of inhibiting infections caused by filamentous fungi, comprising contacting the filamentous fungi with a composition comprising Vibrio gazogenes, ATCC 43942 thereby inhibiting infections caused by the filamentous fungi.

3. A method of disrupting endosomal function in filamentous fungi comprising contacting the filamentous fungi with a composition comprising Vibrio gazogenes, ATCC 43942 thereby disrupting endosomal function in the fungal cell.

4. A method of restricting hyphal fusion formation in filamentous fungi, comprising contacting the filamentous fungi with a composition comprising Vibrio gazogenes, ATCC 43942 thereby restricting hyphal fusion formation in the fungal cell.

5. The method of claim 1, wherein the Vibrio gazogenes, ATCC 43942 is dead.

6. The method of claim 2, wherein the Vibrio gazogenes, ATCC 43942 is dead.

7. The method of claim 3, wherein the Vibrio gazogenes, ATCC 43942 is dead.

8. The method of claim 4, wherein the Vibrio gazogenes, ATCC 43942 is dead.

9. The method of claim 5, further comprising heating the composition comprising Vibrio gazogenes, ATCC 43942 at a suitable temperature and for a suitable time prior to contacting the composition with filamentous fungi to kill the Vibrio gazogenes, ATCC 43942.
